SEQUENCE LISTING (7 sequences)

SEQ ID NO: 1 - protein, 14 aa, Homo sapiens
VHLTPEEKSAVTAC

SEQ ID NO: 2 - protein, 14 aa, Homo sapiens
VHLTPVEKSAVTAC

SEQ ID NO: 3 - protein, 14 aa, Homo sapiens
VHLTPKEKSAVTAC

SEQ ID NO: 4 - protein, 20 aa, Homo sapiens
CEFTPPVQAAYQKVVAGVAN

SEQ ID NO: 5 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 6 - protein, 147 aa, Homo sapiens [sequence omitted]

SEQ ID NO: 7 - protein, 147 aa, Homo sapiens [sequence omitted]

(19) United States
(12) Patent Application Publication
(10) Pub. No.: US 2024/0361342 A1
(43) Pub. Date: Oct. 31, 2024

(54) LATERAL FLOW IMMUNOASSAY METHOD OF SIMULTANEOUSLY DETECTING HEMOGLOBIN S, HEMOGLOBIN C, AND HEMOGLOBIN A IN NEWBORNS, INFANTS, CHILDREN, AND ADULTS

(71) Applicant: BioMedomics, Inc., Morrisville, NC (US)

(72) Inventors: YiTing Cao, Durham, NC (US); XiaoXi Yang, Durham, NC (US); XueFeng Wang, Chapel Hill, NC (US)

(73) Assignee: BioMedomics, Inc., Morrisville, NC (US)

(21) Appl. No.: 18/662,553

(22) Filed: May 13, 2024

Related U.S. Application Data
(62) Division of application No. 14/725,894, filed on May 29, 2015.
(60) Provisional application No. 62/067,702, filed on Oct. 23, 2014.

(51) Int. Cl. G01N 33/72; C07K 14/805; C07K 16/18; G01N 33/531

(52) U.S. Cl. CPC G01N 33/721; C07K 14/805; C07K 16/18; G01N 33/531; G01N 33/726; C07K 2317/34; G01N 2333/805

(57) ABSTRACT

Screening methods and devices for detecting and diagnosing hemoglobinopathies for sickle cell disease and related phenotypes. Lateral flow immunoassay devices for the detection of hemoglobinopathies. Methods for screening for hemoglobinopathies. Kits for the detection of a hemoglobinopathy in a sample. Immunogenic peptides for producing antibodies against hemoglobin variants.

### CROSS-REFERENCE TO RELATED APPLICATIONS

This application is a divisional of U.S. patent application Ser. No. 14/725,894, filed May 29, 2015, which claims the benefit of and priority to U.S. Provisional Patent Application Ser. No. 62/067,702, filed Oct. 23, 2014. The entire disclosures of both of these applications are herein incorporated by reference in their entireties.

### REFERENCE TO SEQUENCE LISTING XML

The Sequence Listing XML associated with the instant disclosure has been electronically submitted to the United States Patent and Trademark Office via the Patent Center as a 7,627 byte UTF-8-encoded XML file created on May 13, 2024 and entitled “3108_3_3_DIV.xml”. The Sequence Listing submitted via Patent Center is hereby incorporated by reference in its entirety.

### TECHNICAL FIELD

The present disclosure relates to hematology, reagents and methods of detecting blood diseases in samples, such as for example samples of whole blood, packed red cells, and/or dry blood spots. The present disclosure also relates to methods for screening for sickle cell diseases and traits, hemoglobin C disease and traits, and β-thalassemia hemoglobinopathies to diagnose diseases and provide information for therapeutic guidance, evaluation and monitoring. More particularly, the present disclosure relates to immunoassays for screening of hemoglobin A, hemoglobin S and hemoglobin C hemoglobinopathies. Also disclosed are reagent combinations and kits for use in such assays.

### BACKGROUND

Human hemoglobins (Hbs) are tetramers of globin chains folded around four heme groups. A functional hemoglobin (Hb) is composed of two alpha (α) globin-chains and two non-alpha (beta, gamma or delta) globin-chains. A total of eight functional globin chains are found in various stages of development, producing eight types of normal Hb tetramers. Adult humans have predominantly HbA (“normal” or “common” hemoglobin) and a small amount of HbA₂. The Hb of a newborn is comprised of about 60-85% fetal hemoglobin (HbF) and about 15-40% HbA. Newborn Hb comprises only a barely detectable level of HbA₂.

As a result of mutations in the genes that encode the different Hb chains, there are more than 800 known variants of normal Hbs. The majority of these variants are due to substitutions of amino acids on a single globin-chain. Most of the mutations produce no clinically significant abnormal Hb function, e.g. impaired oxygen uptake or carbon dioxide release. However, some Hb variants do cause severe disease conditions and are thus called hemoglobinopathies.

The variant Hbs are geographically unevenly distributed all over the world. Hemoglobinopathies with high incidence and prevalence have become a serious healthcare threat and social-economic problem in many countries. The earliest identified and clinically most significant variant is sickle cell disease (SCD), which is caused by a single amino acid substitution at the sixth position from the N-terminus of the beta-globin chain ((β6^{Glu→Val}). Sickle-cell disease or sickle-cell anemia (SCA) is a hereditary blood disorder characterized by rigid sticky red blood cells (RBCs) with a sickle or crescent shape. Sickling decreases the RBC flexibility and results in various complications. Sickle cell anemia (homozygous HbSS) is the result of beta-globin genes from both parents having the sickle cell mutation that are inherited by the patient. The World Health Organization (WHO) has declared SCD an epidemic and public health priority (1). The HbS variant is traditionally most common in populations of African, Indian and Mediterranean ethnicity. Due to increased ethnic diversity and worldwide immigration, hemoglobinopathies have become more common in Europe, the United States and South America than before. The greatest burden of SCD is in sub-Saharan Africa, where 75% of the 300,000 annual global births of affected children live. Estimates suggest that approximately 20% of children with SCD die within their first two years, often by opportunistic infections to the lungs. In addition, 50-80% of these patients will die before reaching adulthood. An early identification of the HbS mutation substantially decreases the mortality and morbidity during the first five years of life if those identified soon after birth are given prophylactic treatments. Individuals with heterozygote (HbAS) show no such symptoms but still have the risk to pass the β6^{Glu→Val} mutation to their offspring. The WHO estimates that 70% of SCD deaths in Africa are preventable with simple, cost-effective interventions such as early identification of SCD patients by newborn screening (NBS) and subsequent provision of comprehensive care (2). Unfortunately, in many developing countries and in rural areas, SCD tests are rarely made before children reach the age of 2-3 (3). This delayed SCD test results in late diagnosis and/or misdiagnosis, severe complications, and poor prognosis. The reason for delayed SCD test is the lack of sensitive and specific point-of-care (POC) device for the diagnosis of SCD in low resource settings. Traditional tests including zone electrophoresis, isoelectric focusing (IEF) electrophoresis, high-performance liquid chromatography (HPLC) and DNA analysis are complicated, cumbersome, relying on expensive equipment and professional technologists.

Hemoglobin C (HbC) is an abnormal hemoglobin in which substitution of a glutamic acid residue with a lysine residue at the sixth position of the β-globin chain has occurred (β6^{Glu→Lys} mutation). Similar to sickle cell mutation, this mutation reduces the normal plasticity of red blood cells causing a hemoglobinopathy. In those who are heterozygous for the mutation, about 28 to about 44% of total Hb is HbC, and no anemia develops. In homozygotes, nearly all Hb is in the HbC form, resulting in mild hemolytic anemia. Individuals with sickle cell-hemoglobin C (HbSC) have the gene for HbS inherited from one parent and the gene for HbC is inherited from the other parent, i.e. they are heterozygous. Since HbC does not polymerize as readily as HbS there is less sickling (fewer sickle cells). The peripheral smear demonstrates mostly target cells and only a few sickle cells. There are fewer acute vaso-occlusive events. However, persons with hemoglobin SC disease (HbSC) have more significant retinopathy, ischemic necrosis of bone, and priapism than those with pure SS disease. People with hemoglobin C trait have red blood cells that have normal hemoglobin A and an abnormal hemoglobin C. People with hemoglobin C trait have slightly more hemoglobin A than hemoglobin C. People with Hemoglobin C trait do not have health problems related to having the trait. People with hemoglobin C trait do not have Hemoglobin C disease or sickle cell disease. They cannot develop these diseases later in life. However, they can pass hemoglobin C trait to their offspring. Individuals who carry the hemoglobin C trait can have a child with Hemoglobin C disease or Hemoglobin SC disease.

Hemoglobin C gene is found in about 2% to about 3% of African Americans in the United States while the percentage for hemoglobin S (sickle) gene is about 8% in African Americans in the United States. Therefore, Hemoglobin SC disease is significantly more common than Hemoglobin CC disease. About 1 out of every 40 African Americans has the hemoglobin C trait. People whose ancestors came from Italy, Greece, Africa, Latin America, and the Caribbean region have higher possibility to have hemoglobin C gene although it is possible for a person of any race or nationality to have hemoglobin C trait. Hemoglobin C disease is present at birth, though some cases may not be diagnosed until adulthood. Both male and female are affected equally.

HbS occurs at such a high frequency that routine screening of newborns to identify possibly afflicted subjects is recommended in most developed nations. In some areas, such as sub-Saharan Africa, Central India and Brazil, newborn babies should be subjected to neonatal testing for a possible SCD, sickle cell trait, HbC disease, or HbC trait but are not due to access to laboratory technologies.

There is thus an established need for inexpensive and easy-to-use screening assays, devices and methods which diagnose, or distinguish a healthy subject from a possibly afflicted subject needing further testing to diagnose a possible sickle cell and/or Hemoglobin C hemoglobinopathy. In addition, so far there are no POC tests that can simultaneously qualitatively and/or quantitatively detect HbS, HbC, and HbA. Therefore, the current sickle cell tests can neither distinguish HbSS from HbSC, nor distinguish HbCC from HbAC.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments provided herein are immunoassay systems. Such immunoassay systems can in some embodiments comprise a capture antibody having a binding affinity to human hemoglobin A (HbA), human sickle cell hemoglobin (HbS), or human hemoglobin C (HbC), wherein a capture antibody having an affinity to HbA comprises an antibody having an affinity to an amino acid sequence of SEQ ID NO. 1, wherein SEQ ID NO. 1 comprises a 14 amino acid sequence of the N-terminus of wild-type human hemoglobin β-chain, wherein a capture antibody having an affinity to HbS comprises an antibody having an affinity to an amino acid sequence of SEQ ID NO. 2, wherein SEQ ID NO. 2 comprises a 14 amino acid sequence of the N-terminus of a mutated human hemoglobin β-chain, and wherein a capture antibody having an affinity to HbC comprises an antibody having an affinity to an amino acid sequence of SEQ ID NO. 3, wherein SEQ ID NO. 3 comprises a 14 amino acid sequence of the N-terminus of a mutated human hemoglobin α-chain, and a conjugated detector antibody, wherein the detector antibody has a binding affinity to hemoglobin (Hb), wherein the detector antibody is conjugated to a detectable moiety.

In some embodiments, the assay system can be configured as an enzyme-linked immunosorbent assay (ELISA), a flow cytometry assay, a competitive immunoassay, a noncompetitive immunoassay, a radioimmunoassay, a chemiluminescent immunoassay, a fluorogenic immunoassay or a colormetric immunoassay. In some embodiments, the assay system can further comprise a substrate upon which the capture antibody can be immobilized, wherein the substrate can comprise a chromatography matrix, a polymer surface, a bead or a multiwell plate. In some embodiments, the assay system can further comprise a capture antibody for each of HbA, HbS and HbC.

In some embodiments, provided herein is a lateral flow immunoassay device for the detection of hemoglobinopathies, comprising a lateral flow test strip comprising a chromatography matrix, a capture antibody immobilized on the test strip, wherein the capture antibody has a binding affinity to human hemoglobin A (HbA), human sickle cell hemoglobin (HbS), or human hemoglobin C (HbC), and a conjugated detector antibody, wherein the detector antibody has a binding affinity to hemoglobin (Hb), wherein the detector antibody is conjugated to a detectable moiety. In some embodiments, the device further comprises a first capture antibody immobilized on the test strip, wherein the first capture antibody has a binding affinity to HbA, a second capture antibody immobilized on the test strip, wherein the second capture antibody has a binding affinity to HbS, and a third capture antibody immobilized on the test strip, wherein the third capture antibody has a binding affinity to HbC.

In some embodiments, the first capture antibody having an affinity to HbA comprises an antibody having an affinity to an amino acid sequence of SEQ ID NO. 1, wherein SEQ ID NO. 1 comprises a 14 amino acid sequence of the N-terminus of wild-type human hemoglobin β-chain, wherein the second capture antibody having an affinity to HbS comprises an antibody having an affinity to an amino acid sequence of SEQ ID NO. 2, wherein SEQ ID NO. 2 comprises a 14 amino acid sequence of the N-terminus of a mutated human hemoglobin β-chain, and wherein the third capture antibody having an affinity to HbC comprises an antibody having an affinity to an amino acid sequence of SEQ ID NO. 3, wherein SEQ ID NO. 3 comprises a 14 amino acid sequence of the N-terminus of a mutated human hemoglobin β-chain. In some embodiments, the first, second and third capture antibodies are immobilized on the test strip in analyte capture zones.

In some embodiments, the immunoassay device further comprises a fourth capture antibody immobilized on the test strip in a fourth analyte capture zone, wherein the fourth capture antibody has an affinity to IgG of the host animal of the conjugate detector antibody. In some embodiments, the fourth capture antibody serves as a control for the immunoassay. In some embodiments, the analyte capture zones are rectangular shaped or circular shaped. In some embodiments, the analyte capture zones are simplexed or multiplexed with one or more antibodies having an affinity for HbA, HbS, HbC, other hemoglobins (Hbs), or combinations thereof.

In some embodiments, the test strip further comprises a sample receiving area. In some embodiments, the sample receiving area is configured to receive whole blood samples, dried blood samples, packed red cell samples, isolated or purified human hemoglobin protein samples, or freshly collected filter paper samples. In some embodiments, the analyte capture zones are arranged on the test strip in a linear array parallel and substantially equidistant on chromatography matrix.

In some embodiments, the test strip further comprises a conjugate pad, wherein the conjugate pad comprises the conjugated detector antibody, optionally wherein the conjugated detector antibody is impregnated into the conjugate pad. In some embodiments, the conjugate pad is located between the sample receiving area and the analyte capture zones. In some embodiments, the conjugated detector antibody comprises a polyclonal or monoclonal detection antibody against human hemoglobin α-chain or β-chain. In some embodiments, the detector antibody comprises an antibody having an affinity to an amino acid sequence of SEQ ID NO. 4, wherein SEQ ID NO. 4 comprises an amino acid sequence of the C-terminus of wild-type human hemoglobin β-chain.

In some embodiments, the detectable moiety of the conjugated detector antibody comprises an enzyme label, a fluorescent label, a radiolabel, a particulate label, a colloidal gold label, a colored latex particle, or a phosphor converting label. In some embodiments, the chromatography matrix comprises a nitrocellulose membrane, polyvinylidene fluoride membrane, (charge-modified) nylon membrane, or polyethersulfone membrane. In some embodiments, the lateral flow test strip comprises a component of a competition assay, an indirect assay or a sandwich assay. In some embodiments, the immunoassay device is configured to simultaneously detect HbS, HbC, HbA or other hemoglobin variants in a sample. In some embodiments, the immunoassay device is configured to quantify the levels of HbS, HbC, HbA or other hemoglobin variants in absolute or relative terms. In some embodiments, wherein the immunoassay device is configured to assist in diagnosing hemoglobinopathies. In some embodiments, the immunoassay device is configured to be used at the point-of-care.

In some embodiments, provided herein is a method for screening for hemoglobinopathies, comprising providing a blood sample, mixing the blood sample with a buffer, providing the lateral flow immunoassay device, and loading the blood sample mixed in the buffer on the immunoassay device, wherein a hemoglobinopathy present in the sample will be revealed by the lateral flow immunoassay device. In some embodiments, the presence of HbS, HbC, and/or HbA in the sample is simultaneously determined. In some embodiments, the level of HbS, HbC, and/or HbA is quantified in absolute or relative terms. In some embodiments, the method comprises simultaneously differentiating sickle cell disease and trait, HbC disease and trait, and normal human hemoglobin, based on the presence of one or more of HbS, HbC, and/or

HbA. In some embodiments, the hemoglobinopathy is selected from the group consisting of: HbAS, HbSS, HbSB+-thalassemia, HbSβ⁰-thalassemia, sickle hemoglobin C disease (HbSC), and HbSX, wherein X is a globin chain variant that is not HbA, selected from the group consisting of HbSD-Punjab, HbSO-Arab, HbFS and HbSE. In some embodiments, the hemoglobinopathy is selected from a hemoglobin C disease or hemoglobin C trait, selected from the group consisting of HbC disease (HbCC), HbCβ⁰-thalassemia, HbCβ⁺-thalassemia, hemoglobin C trait (HbAC). In some embodiments, the blood sample is selected from the group consisting of whole blood sample, dried blood sample, packed red cell sample, isolated or purified human hemoglobin protein sample, and freshly collected filter paper sample. In some embodiments, providing a blood sample comprises finger-stick, heel-stick or venipuncture. In some embodiments, mixing the blood sample with a buffer results in hemolysis, dilution and conditioning of the sample. In some embodiments, loading the blood sample mixed in the buffer on the assay device comprises adding the sample to a sample receiving area of the device.

In some embodiments, provided herein is an immunogenic peptide capable of eliciting a human HbC, HbS or HbA specific polyclonal or monoclonal antibody, the peptide comprising the first 13 amino acids from the N-terminus of HbC, HbS or HbA, wherein the peptide comprises an amino acid sequence selected from SEQ ID NO. 1, SEQ ID NO. 2, or SEQ ID NO. 3. In some embodiments, the immunogenic peptide further comprises a cysteine at the fourteenth amino acid to facilitate peptide conjugation. In some embodiments, provided is a polyclonal or monoclonal antibody capable of binding the immunogentic peptide.

In some embodiments, provided herein is an immunogenic peptide capable of eliciting a human hemoglobin α-chain or β-chain specific polyclonal or monoclonal antibody, the peptide comprising the first 18 amino acids from the C-terminus of human hemoglobin α-chain or β-chain, wherein the peptide comprises an amino acid sequence of SEQ ID NO. 4. In some embodiments, the immunogenic peptide further comprises a cysteine at the first amino acid to facilitate peptide conjugation.

In some embodiments, provided is a polyclonal or monoclonal antibody capable of binding the immunogentic peptide. In some embodiments, provided is a method of generating a polyclonal or monoclonal antibody having an affinity to human HbC, HbS, HbA or other hemoglobin variants, comprising administering an immunogenic peptide to immunize a subject, collecting serum of the subject, and purifying and depleting antibodies generated in the collected serum. In some embodiments a polyclonal or monoclonal antibody generated by this method is provided.

In some embodiments, provided is a method of generating a polyclonal or monoclonal antibody having an affinity to human hemoglobin α-chain or β-chain, comprising administering an immunogenic peptide to immunize a subject, collecting serum of the subject, and purifying and depleting antibodies generated in the collected serum. In some embodiments, provides is polyclonal or monoclonal antibody generated by this method.

In some embodiments, provided is a kit for the detection of a hemoglobinopathy in a sample, comprising a device, a sampler for collecting a blood sample, a buffer module containing a buffer, and instructions for performing the detection of a hemoglobinopathy. In some embodiments, the sampler for collecting a blood sample comprises a capillary tube. In some embodiments, the buffer module containing a buffer comprises a two-piece cap. In some embodiments, the buffer comprises an extraction buffer with a detergent.

Accordingly, it is an object of the presently disclosed subject matter to provide methods, devices, systems and kits for screening and/or assisting in diagnosing hemoglobinopathies. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a schematic illustration of a test strip configured to be used in a simplexed or multiplexed lateral flow immunoassay.

FIGS. 2A-2H are images of lateral flow immunoassays depicting results from subjects with differing hemoglobinopathy phenotypes.

FIGS. 3A-3D are schematic illustrations of immunogenic peptides for eliciting antibodies against HbA, HbS, HbC and hemoglobin β-chain, respectively.

FIGS. 4A-4D provide a schematic illustration of a kit and process for screening a sample for hemoglobinopathies.

FIGS. 5A-5E are images of lateral flow immunoassays depicting results from subjects with differing hemoglobinopathy phenotypes.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

The present disclosure provides in some embodiments a lateral flow immunoassay (LFIA), sometimes referred to as multiplexed-line lateral flow immunochromatographic assay (MLFIA), configured to simultaneously, qualitatively and/or quantitatively, detect HbS, HbC, and HbA in a sample. In some aspects, the present disclosure provides for the design, selection, production, and usage of specific polyclonal antibodies (pAbs) and/or monoclonal antibodies (mAbs) having an affinity to HbS, HbC, and HbA. Also disclosed herein is the conjugation of pAbs and/or mAbs to colored or fluorescent nanoparticles for detection and/or visualization of HbS, HbC, and HbA in an assay, such as for example a POC LFIA. Moreover, in some embodiments the present disclosures provides methods, kits and systems for screening for hemoglobinopathies in subjects, such as human subjects, by obtaining and/or collecting blood samples and detecting the presence of HbS, HbC, and/or HbA in the samples.

In some aspects, the present disclosure provides a method to detect hemoglobinopathies on human hemoglobin β-chain using whole blood samples. A polyclonal or monoclonal antibody against the C-terminus of human hemoglobin α-chain or β-chain can be used as the detection antibody. This detection antibody can be conjugated to colored (or fluorescent) nanoparticles. Other pAbs and/or mAbs against the initial N-terminal amino acid (AA) sequence of human sickle cell hemoglobin (HbS), human hemoglobin C (HbC), and adult normal hemoglobin (HbA) can be used as capture antibodies. These capture antibodies can in some aspects be incorporated as multiplexed test lines designated as “S”, “C”, and “A” on a nitrocellulose membrane and can specifically bind each targeted hemoglobin (Hb) from human whole blood sample. The resulting colored “S”, “C”, and “A” lines on the nitrocellulose membrane can be used to qualitatively or quantitatively determine various HbS, HbC hemoglobinopathies and their traits including, but not limited to, normal hemoglobin A (HbA), sickle cell trait (HbAS); sickle cell disease (including HbSS, HbSβ⁰-thalassemia, HbSβ⁺-thalassemia, and HbSC); hemoglobin C diseases (HbCC) and their traits (HbAC), and adult normal hemoglobin (HbAA), etc.

The disclosed assays, POC devices, methods, kits and systems differ from previous tests for determining the presence or absence of HbS and/or HbC in whole blood sample, and offer significant advantages not realized in the prior art. For example, solubility testing methods such as Sickledex® (Streck, Inc., Omaha, Nebraska, United States of America) and concentrated phosphate buffer are simple and inexpensive, but do not differentiate between SCD (including HbSS, HbSβ⁰-thalassemia, HbSβ⁺-thalassemia, and HbSC) and Sickle-Cell Trait (HbAS). The methods are based on HbS polymerization (visible turbid suspension) in the presence of a concentrated phosphate buffer solution. All positive test results should be further evaluated by hemoglobin electrophoresis or HPLC, when used for patient testing. The current solubility test fails to detect HbS in persons with severe anemia, and in infants under six months of age due to elevated levels of hemoglobin F. These deficiencies are overcome by the disclosed methods and testing apparatuses.

In an optimized solubility testing method, instead of measuring turbidity, the characteristic blood stain formed on a paper-based assay becomes an active element (4, 5). The polymerized HbS is entangled by the paper fibers. The soluble hemoglobin will continue to spread on the paper and, because it is colored, the assay read out uses the red color count in the region of the polymerized hemoglobin and soluble hemoglobin. The visual signals need to be analyzed by a scanner to correlate the blood stain pattern with the concentration of HbS present. But this assay cannot accurately distinguish individuals with HbAS (trait) and HbSC (disease) since they have similar HbS concentration. In a person with HbSC, the presence of HbC enhances the pathogenic properties of HbS by inducing dehydration and therefore sickling at a significant level that would not take place in a person with similar levels of HbS. Here again, these deficiencies are overcome by the disclosed methods and testing apparatuses.

A hemolysis monitoring assay in non-electrolyte solutions (6) is proposed to distinguish red blood cells from HbSS and HbAS individuals based on the altered properties of the RBC membrane resulting from HbS polymerization. However, an hour incubation time, the use of tonometer and optical density measurements make the proposed test difficult to be used at POC. The recent development of cell density based aqueous multiphase system (7, 8) requires a drop of whole blood, which goes into a capillary tube filled with three different polymeric aqueous solutions. After centrifugation in a small, battery-operated instrument, sickle cells are separated from normal red blood cells, based on differences in their cell density. The isolated sickle cells fraction needs to be detected by a simple optical reader. The use of centrifuge and optical reader challenges the simplicity of the POC test. Additionally, HbAA (normal) and HbAS (trait) have the same performance by using this system, and cannot be distinguished. These deficiencies are overcome by the disclosed methods and testing apparatuses.

The instant disclosure overcomes the above-noted deficiencies in existing testing methods, at least in part, by the use of an immunoassay platform that incorporates multiple pAbs and/or mAbs to HbS, HbC, and HbA. These pAbs and/or mAbs act as capture antibodies and are incorporated or immobilized into a low-cost LFIA POC test cassette. In some embodiments, the multiplexed innovation provides: 1) high specificity to HbS, HbC, and HbA 2) high sensitivity by identifying HbS, HbC, and HbA in the presence of elevated HbF, and 3) identifying a patient's phenotype as normal (HbAA), or sickle trait (heterozygous HbAS), or heterozygous HbAC from those with SCD (HbSS, HbS β-thalassemias, HbSC). Such a rapid POC multiplexed test allows basic health care workers to perform the assay in the field and to screen for and/or identify those patients with SCD, or believed to have SCD, quickly and at low cost.

### Immunoassay Systems and Devices

In some embodiments provided herein are immunoassay systems and/or devices. Such immunoassays can in some aspects comprise a capture antibody having a binding affinity to a hemoglobin, such as for example HbA, HbS, and/or HbC. In some embodiments a capture antibody having an affinity to HbA can comprise an antibody having an affinity to an amino acid sequence of SEQ ID NO. 1, wherein SEQ ID NO. 1 can comprise a 14 amino acid sequence of the N-terminus of wild-type human hemoglobin β-chain. In some embodiments a capture antibody having an affinity to HbS can comprise an antibody having an affinity to an amino acid sequence of SEQ ID NO. 2, wherein SEQ ID NO. 2 can comprise a 14 amino acid sequence of the N-terminus of a mutated human hemoglobin β-chain. In some embodiments a capture antibody having an affinity to HbC can comprise an antibody having an affinity to an amino acid sequence of SEQ ID NO. 3, wherein SEQ ID NO. 3 can comprise a 14 amino acid sequence of the N-terminus of a mutated human hemoglobin β-chain.

In some aspects a conjugated detector antibody can have a binding affinity to hemoglobin (Hb), wherein the detector antibody can be conjugated to a detectable moiety. In some aspects an assays system can include a capture antibody for each of HbA, HbS and HbC. Such immunoassays can be configured in a variety of formats and/or platforms. By way of example and not limitation, such configurations can comprise an enzyme-linked immunosorbent assay (ELISA), a flow cytometry assay, a competitive immunoassay, a noncompetitive immunoassay, a radioimmunoassay, a chemiluminescent immunoassay, a fluorogenic immunoassay and/or a colormetric immunoassay. Moreover, in some aspects an assay system can comprise a substrate upon which the capture antibody can be immobilized. By way of example and not limitation, the substrate can comprise a chromatography matrix, a bead or a multiwell plate.

### Lateral Flow Immunoassay (LFIA) Devices

In some embodiments lateral flow immunoassay devices are provided for the detection of HbA, HbS and/or HbC in a sample. In some aspects these immunoassay devices can detect hemoglobinopathies in a sample from a subject. Such a LFIA device can in some aspects comprise a lateral flow test strip comprising a chromatography matrix, a capture antibody immobilized on the test strip, and a detector antibody, such as a conjugated detector antibody. The capture antibody can have a binding affinity to HbA, HbS and/or HbC. The conjugated detector antibody can have a binding affinity to hemoglobin (Hb), and the detector antibody can be conjugated to a detectable moiety. In some aspects, such an immunoassay device can comprise a plurality of capture antibodies, including for example a first capture antibody immobilized on the test strip, wherein the first capture antibody has a binding affinity to HbA, a second capture antibody immobilized on the test strip, wherein the second capture antibody has a binding affinity to HbS, and a third capture antibody immobilized on the test strip, wherein the third capture antibody has a binding affinity to HbC.

Because there are several types of SCD (e.g., HbSS, HbSC disease, HbSβ⁺-thalassemia, etc.), simultaneous detection of the presence and/or relative levels of HbS, HbC, and HbA can in some instances be relevant to accurately differentiate various forms of SCD and sickle cell traits. As such, in some embodiments disclosed herein are multiplexed lateral flow immunoassays that are configured to accurately differentiate the most common types of sickle cell conditions. The present disclosure uses in some embodiments a LFIA test cassette that can be used as a qualitative screening test by visualizing the presence or absence, or the different levels of color intensity of the three colored, e.g. blue colored, test lines for HbS, HbC, and HbA on the POC cassette device. Alternatively, or in addition, in some embodiments it can also be used as a quantitative test by the use of a colorimetric or fluorescent reader to measure and compare the color or fluorescence intensities of the above three test lines. The components of a lateral flow assay can in some aspects include a nitrocellulose membrane, a conjugate pad, a detection polyclonal or monoclonal antibody conjugated to colored or fluorescent nanoparticles, polyclonal or monoclonal capture antibodies dispensed as the test lines and the control line, and a wicking pad (absorbent pad).

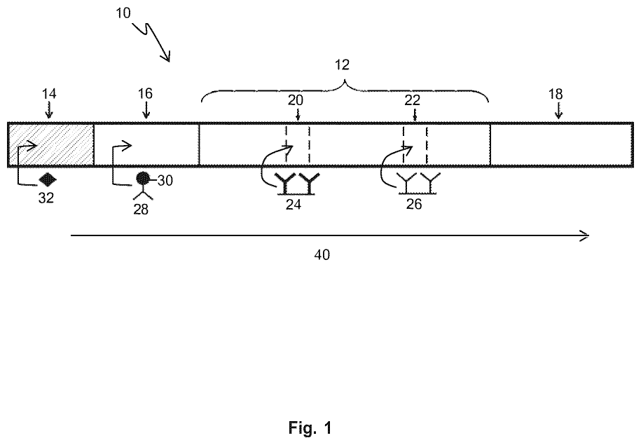

In some embodiments, a LFIA device for the detection of hemoglobinopathies can comprise a lateral flow test strip 10 as depicted in FIG. 1. Test strip 10 can comprise a chromatography matrix 12, such as for example a nitrocellulose membrane, polyvinylidene fluoride membrane, (charge-modified) nylon membrane, polyethersulfone membrane. In some embodiments, test strip 10 can comprise a sample pad 14 and conjugate pad 16. In some embodiments, test strip 10 can further comprise an absorbent pad 18. In some aspects, sample pad 14, conjugate pad 16, and/or absorbent pad 18, can be built into chromatography matrix 12, or can be adjacent to but separate from chromatography matrix 12.

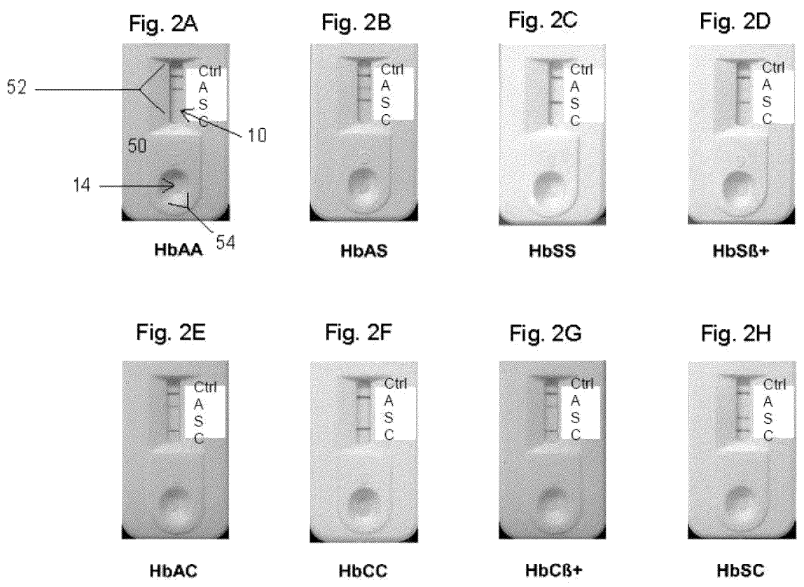

Continuing with FIG. 1, test strip 10 can further comprise one or more test lines or analyte capture zones 20. In some embodiments, an analyte capture zone 20 can comprise one or more capture antibodies 24 having an affinity to an analyte, e.g. a protein or peptide. In some aspects, analyte capture zone 20 can be rectangular shaped, circular shaped or any other shape, and where multiple analyte capture zones 20 are present each is spaced apart along chromatography matrix 12 to create discrete analyte capture zones. Such a simplexed configuration (see, e.g., FIGS. 2A-2H) can provide for the detection of multiple analytes simultaneously using multiple capture antibodies 24.

Continuing with FIG. 1, in some embodiments, test strip 10 can comprise a control analyte capture zone 22 comprising a capture antibody 26 having an affinity to an analyte or antigen that is expected to always be present in a sample from a subject. Control analyte capture zone 22 can therefore be configured to act as a control that indicates the assay is working properly. Capture antibody 26 can have an affinity to IgG of host animals 28, detectable moiety 30 or one or multiple protein, peptide, chemical or other ingredients in the test sample 32.

As depicted in FIG. 1, sample pad 14 can comprises a sample receiving area configured to receive a sample 32, wherein a sample 32 can comprise any sample from a subject, e.g. a human, that contains, is believed to contain, or can contain hemoglobin. Samples to be taken from subjects and analyzed using the disclosed immunoassay devices and methods can comprise, for example, whole blood samples, dried blood samples, packed red cell samples, isolated or purified human hemoglobin protein samples, or freshly collected filter paper samples.

In some embodiments, test strip 10 can comprise a conjugate pad 16 having impregnated thereon, or otherwise releasably attached thereto, a conjugated detector antibody 28 having conjugated, or otherwise joined thereto, a detectable moiety 30. Conjugated detector antibody 28 can be an antibody having an affinity to the analyte or antigen captured by one or more capture antibodies 24 on the test strip. By virtue of the detectable moiety 30, the presence of a captured analyte or antigen of interest can be visible or discernable. Detectable moiety 30 can comprise any detectable compound that can be suitably conjugated or adjoined to detector antibody 28. By way of example and not limitation, the detectable moiety 30 of conjugated detector antibody 28 can comprises an enzyme label, a fluorescent label, a radiolabel, a particulate label, a colloidal gold label, a colored latex particles, a phosphor converting label, dyes, chromophores, affinity probes, groups with specific reactivity, chemiluminescent moieties, and/or electrochemically detectable moieties. In some embodiments, conjugate pad 16 is located between sample pad 14 and analyte capture zone(s) 20.

Continuing with FIG. 1, in some embodiments test strip 10 is configured such that the addition or application of a sample 32 to sample pad 14 will cause the lateral movement, flow or wicking of the contents of sample 32, including any analytes and/or antigens of interest, from the sample pad 14, through conjugate pad 16, and across analyte capture zone(s) 20 and control analyte capture zone 22. This lateral flow 40 or movement of the sample contents is illustrated in FIG. 1. Flow 40 can be facilitated by the capillary action of test strip 12, which in some embodiments can be enhanced by absorbent or wicking pad 18. Thus, when in use, an analyte of interest, for example HbA, in a sample can migrate from sample pad 14 to conjugate pad 16 where it can interact with conjugated detector antibody 28. The analyte of interest, now with bound conjugated detector antibody 28, can continue to migrate to analyte capture zone 20 where it can become bound to a capture antibody 24. Then, the presence of the analyte of interest can be detected by virtue of detectable moiety 30 conjugated to conjugated detector antibody 28. The remainder of sample 32 can continue to flow or migrate toward absorbent pad 18, including for example a control analyte which can be captured by control capture antibody 26. As discussed herein, in some embodiments sample 32 can be mixed with a buffer to, among other things, optimize the sample for migration by capillary action down test strip 10 of a LFIA.

To make test strip 10 each polyclonal or monoclonal antibody that is to serve as a capture antibody 26 or control capture antibody 26 can be dispensed and immobilized on a chromatography matrix 12 (such as, but not limited to, a nitrocellulose membrane). One end of this matrix can be overlapped by a piece of absorbent pad 18 (wick pad) and the other end of this matrix 12 can be overlapped by a conjugate pad 14. In some embodiments, the multiplexed antibody lines, or analyte capture zones 20, on the matrix from a position near the absorbent pad 18 to the other position near the conjugated pad 16 can be arranged as follows: Control line, HbA test line, HbS test line, and HbC test line. Of course, other configurations and/or orientations are equally applicable. To apply the capture antibodies 24, 26 to test strip 10 a printing buffer (including sucrose, trehalose and sodium azide in PBS) can be used. In some embodiments, capture antibodies 24, 26 mixed in a printing buffer can be dispensed at the analyte capture zones 20 on the chromatography matrix in amounts of about 5 to about 100 ng/mm, in some aspects about 20 to about 25 ng/mm, in lines (or other desired shape) with a width of about 0.1 to about 0.8 mm. The chromatography matrix can then be dried at about 50° C. for about 15 minutes. The colored or fluorescent nanoparticles conjugated with polyclonal or monoclonal detection antibodies can then be sprayed at a dispense rate of about 1.25 μg/mm detectable moiety in HSTT buffer, immunized on the conjugate pad and allowed to dry overnight at room temperature. In some embodiments, a piece of sample pad 14 can be placed on top of conjugate pad 16 but not connected and touched to the chromatography matrix 12. The chromatography matrix, conjugate pad, sample pad, absorption pad, and the backing card can in some embodiments be assembled and cut to a width of about 4 mm using a cutter so that the final strip had a dimension of about 2 to about 10 mm in width and about 50 to about 100 mm in length. The manufactured strip can be assembled in a disposable cassette and packed in a dehumidifier followed by storage at room temperature until use.

As depicted in FIG. 2A, test strip 10 can be contained or housed within a cassette or housing 50. A cassette 50 can be configured to secure test strip 10 in a rigid structure that provides a portable, easy-to-use, and disposable POC device. Cassette 50 can comprise a sample receiving area 54 adjacent to sample pad 14 on test strip 10 to thereby provide a receptacle for receiving a sample and depositing it directly on to sample pad 14. Cassette 50 can also comprise a window 52 or opening to permit access to and visible observation of analyte capture zones 20 and 22. The remainder of test strip 10 can be enclosed or covered by cassette 50, as depicted in FIG. 2A, for example, or can be exposed or uncovered.

In some embodiments, a LFIA as disclosed herein, and particularly the test strip, can comprise first, second and third capture antibodies, each of which can be immobilized on the test strip in discrete analyte capture zones. In some embodiments, a first capture antibody immobilized on the test strip can have a binding affinity to human hemoglobin A (HbA), while a second capture antibody immobilized on the test strip can have a binding affinity to human sickle cell hemoglobin (HbS), and while a third capture antibody immobilized on the test strip can have a binding affinity to human hemoglobin C (HbC). Thus, in some embodiments, an immunoassay device as disclosed herein can comprise multiple capture antibodies in a multiplexed arrangement, such as a multiplex of antibodies directed against HbA, HbS, HbC, other hemoglobins (Hbs), or combinations thereof.

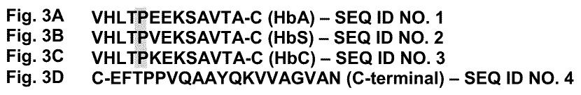

By way of example and not limitation, as depicted in FIG. 2A, a LFIA device 100 can comprise analyte capture zones, or indicators, for each of HbA A, HbS S, HbC C, and control Ctr, on test strip 10. The capture antibody located at the A analyte capture zone (test line) can be a polyclonal or monoclonal antibody specifically against human HbA and/or any protein or peptide containing the sixth position of normal human hemoglobin β-globin chain (SEQ ID NO. 1) and/or its flanking amino acid on both sides as shown in FIG. 3A except for the terminal cysteine (C). The capture antibody located at the S analyte capture zone (test line) can be a polyclonal or monoclonal antibody specifically against human HbS and/or any protein or peptide containing the sixth position point mutation of human hemoglobin β-globin chain (sickle cell mutation: β6^{Glu→Val}; SEQ ID NO. 2) and/or its flanking amino acid on both sides as shown in FIG. 3B except for the terminal cysteine (C). The capture antibody located at the C analyte capture zone (test line) can be a polyclonal or monoclonal antibody specifically against human HbC and/or any protein or peptide containing the sixth position point mutation of human hemoglobin β-globin chain (sickle cell mutation: β6^{Glu→Lys}; SEQ ID NO. 3) and/or its flanking amino acid on both sides as shown in FIG. 3C except for the terminal cysteine (C).

In some embodiments, the first capture antibody having an affinity to HbA can comprise an antibody having an affinity to an amino acid sequence of SEQ ID NO. 1, wherein SEQ ID NO. 1 comprises a 13 amino acid sequence of the N-terminus of wild-type human hemoglobin β-chain with a terminal cysteine, as depicted in FIG. 3A. In some embodiments, the second capture antibody having an affinity to HbS can comprise an antibody having an affinity to an amino acid sequence of SEQ ID NO. 2, wherein SEQ ID NO. 2 comprises a 13 amino acid sequence of the N-terminus of a mutated human hemoglobin β-chain with a terminal cysterine, as depicted in FIG. 3B. In some embodiments, the third capture antibody having an affinity to HbC can comprise an antibody having an affinity to an amino acid sequence of SEQ ID NO. 3, wherein SEQ ID NO. 3 comprises a 13 amino acid sequence of the N-terminus of a mutated human hemoglobin β-chain with a terminal cysteine, as depicted in FIG. 3C.

As depicted in FIG. 3D, the detector antibody employed in a LFIA device or method disclosed herein can comprise an antibody having an affinity to an amino acid sequence of SEQ ID NO. 4, wherein SEQ ID NO. 4 comprises an amino acid sequence of the C-terminus of wild-type human hemoglobin β-chain. In some embodiments, the detector antibody can comprise a polyclonal or monoclonal antibody against human hemoglobin α-chain or β-chain. In some embodiments, the detector antibody employed in a LFIA device as depicted in FIG. 2A, for example, can have an affinity to any analyte, e.g. HbA, HbS, HbC, or other Hbs, such that any analytes captured by the capture antibodies are also tagged with the conjugated detector antibody. Thus, in some aspects a LFIA as disclosed herein can comprise components of a competition assay, an indirect assay and/or a sandwich assay.

As depicted in FIGS. 1 and 2A, in some embodiments the analyte capture zones can be arranged on a test strip in a linear array parallel on chromatography matrix. In some aspects, the analyte capture zones can be spaced substantially equidistant on chromatography matrix.

By way of example and not limitation, in some embodiments a LFIA disclosed herein can comprise multiplexing of up to four antibodies, or more, including for example a first capture antibody against HbC (such as but not limited to β6^{Lys}; SEQ ID NO. 3) on a C test line or analyte capture zone, a second antibody against HbS (such as but not limited to β6^{Val}; SEQ ID NO. 2) on a S test line or analyte capture zone, a third antibody against HbA (such as but not limited to β6^{Glu}; SEQ ID NO. 1) on an A test linear analyte capture zone, and a fourth antibody against IgG of the host animal as a control line. Such a multiplexed immunoassay can allow rapid and easy visualization of the presence or absence of four colored (such as but not limited to blue-colored) reaction lines (three test lines or analyte capture zones for HbC, HbS, HbA, respectively, and one control line), as depicted in FIG. 2A.

In some embodiments, a positive control (Ctrl) line is included to validate the test (see FIGS. 2A through 2H). In addition, the various statuses of C, S, A test lines or analyte capture zones can be used to assist in diagnosing, screening for, and/or differentiating different hemoglobinopathies, as summarized in Table 1.

TABLE 1
Interpretation of MLFIA Results, Diagnosis, and Intervention.

| | | Hemoglobin | | Clinical |
|---|---|---|---|---|
| | Test Line | Status | Diagnosis | Intervention |
| FIG. 2A | Only A | HbAA or | Normal or non-sickle | None |
| | | HbAV | cell disease/trait or | |
| | | | non-HbC disease/trait | |
| FIG. 2B | S and A | HbAS | Sickle-cell trait | None |
| FIG. 2C | Only S | HbSS or Sβ°- | Sickle-cell disease | Penicillin |
| | | thalassemia | | prophy- |
| | | | | laxis |
| FIG. 2D | S, with or | HbSβ⁺- | Sickle-cell disease | Penicillin |
| | without a | thalassemia | | prophy- |
| | little A | | | laxis |
| FIG. 2E | C and A | HbAC | Hemoglobin C trait | None |
| FIG. 2F | Only C | HbCC or | Hemoglobin C | None; note |
| | | HbCβ°- | disease | on report |
| | | thalassemia | | |
| FIG. 2G | C, with or | HbCβ⁺- | Hemoglobin C | None; note |
| | without a | thalassemia | disease | on report |
| | little A | | | |
| FIG. 2H | S and C | HbSC | Sickle-hemoglobin | Penicillin |
| | | | C disease | prophy- |
| | | | | laxis |

Normal, non-sickle cell disease or trait, and non-HbC disease or trait blood samples (i.e. HbAA or HbAX where X represents globin other than HbS, HbC, or HbA) exhibit only a positive A test line (FIG. 2A). Sickle cell trait (HbAS) blood samples show positive at both the S test line and A test line (FIG. 2B). Blood samples from patients with sickle cell disease either show only one positive S test line indicating homozygous HbSS (sickle cell anemia) or HbSβ⁰-thalassemia (FIG. 2C), or show a positive S test line with or without a low-intensity positive A test line indicating HbSβ⁺-thalassemia (FIG. 2D).

The above multiplexed design can also assist in diagnosing, screening for, and/or differentiating three HbC-related conditions: HbAC trait (FIG. 2E), HbC disease (HbCC) or HbSβ⁰-thalassemia (FIG. 2F), and HbSβ⁺-thalassemia (FIG. 2G). To elaborate, hemoglobin C trait (HbAC) blood samples show positive at both S test line and A test line (FIG. 2E). Blood samples from patients with Hemoglobin C disease (HbCC) show either only one positive C test line indicating homozygous HbCC or HbSβ⁰-thalassemia (FIG. 2F), or show a positive C test line with or without a low-intensity positive A test line indicating HbSβ⁺-thalassemia (FIG. 2G). Finally, the disclosed POC LFIA device can also assist in diagnosing, screening for, and/or differentiating sickle-hemoglobin C disease (HbSC). HbSC blood samples show the positive results at both C and S test lines (FIG. 2H).

Thus, as illustrated in FIGS. 2A through 2H, in some embodiments the disclosed immunoassay devices and methods are configured to simultaneously detect HbS, HbC, and HbA in a sample. This can be advantageous in some aspects since there are several types of SCD (e.g., HbSS, HbSC disease, HbSβ⁺-thalassemia, etc.). Therefore, in some embodiments the simultaneous detection of the presence and/or relative levels of HbS, HbC, and HbA can be relevant to accurately differentiate various forms of SCD and sickle cell traits. Thus, the disclosed multiplexed immunoassay devices and methods can be configured to accurately differentiate the most common types of sickle cell conditions. In some embodiments, the disclosed immunoassay devices and methods are configured to have high sensitivity and specificity toward normal and mutant adult human hemoglobin β-chains, including HbS, HbC, and HbA. Representative antibodies showing sufficiently high specificity and sensitivity against normal and mutant adult human hemoglobin β-chains were developed as disclosed herein.

In addition to detecting the presence of HbS, HbC, and/or HbA, in some embodiments the disclosed POC LFIA device is configured to quantify the levels of HbS, HbC, and HbA in absolute or relative terms. For example, a colorimetric or fluorescent reader and/or scanner can be used to measure the color or fluorescence intensities of test lines or analyte capture zones on the test strip by using a pre-created calibration curve between color intensities and percentage of Hbs.

Prior to the instant disclosure there has not been available a SCD diagnostic and/or screening product or method such as disclosed herein. The disclosed first of its kind POC device is configured to diagnose, assist in diagnosing, screen for, and/or differentiate the most common forms of SCD and traits as well as HbC disease and traits. In addition, it is sensitive, specific, rapid, low-cost, easy-to-use, and configured for POC use.

Summarily, the disclosed POC LFIA device for the detection of hemoglobinopathies can in some embodiments provide for high specificity in identifying the presence of HbS, HbC, and HbA in a sample, even in the presence of high-level HbF or HbA2. In some aspects, the disclosed LFIA device is configured to have high sensitivity to simultaneously detect HbS, HbC, and HbA, even in anemic patients. In some embodiments, the disclosed LFIA device is configured with unprecedented capacity to differentiate SCD (homozygous HbSS, heterozygous HbSC, and HbSβ-thalassemias) from sickle cell trait (heterozygous HbAS) and normal adult hemoglobin (HbAA).

### Polyclonal Antibodies (pAbs) and Monoclonal Antibodies (mAbs) Against HbS, HbC, HbA, or the C-Terminus of Hemoglobin α-Chain or β-Chain

TABLE 2
Full length amino acid sequence
The amino-acid sequence of human hemoglobin A (HbA) beta chain (SEQ ID NO. 5):

| | | | | | |
|---|---|---|---|---|---|
| 10 | 20 | 30 | 40 | 50 | 60 |
| MVHLTPEEKS | AVTALWGKVN | VDEVGGEALG | RLLVVYPWTQ | RFFESFGDLS | TPDAVMGNPK |
| 70 | 80 | 90 | 100 | 110 | 120 |
| VKAHGKKVLG | AFSDGLAHLD | NLKGTFATLS | ELHCDKLHVD | PENFRLLGNV | LVCVLAHHFG |
| 130 | 140 | | | | |
| KEFTPPVQAA | YQKVVAGVAN | ALAHKYH | | | |

The amino-acid sequence of human hemoglobin S (HbS)beta chain (SEQ ID NO. 6):

| | | | | | |
|---|---|---|---|---|---|
| 10 | 20 | 30 | 40 | 50 | 60 |
| MVHLTPVEKS | AVTALWGKVN | VDEVGGEALG | RLLVVYPWTQ | RFFESFGDLS | TPDAVMGNPK |
| 70 | 80 | 90 | 100 | 110 | 120 |
| VKAHGKKVLG | AFSDGLAHLD | NLKGTFATLS | ELHCDKLHVD | PENFRLLGNV | LVCVLAHHFG |
| 130 | 140 | | | | |
| KEFTPPVQAA | YQKVVAGVAN | ALAHKYH | | | |

The amino-acid sequence of human hemoglobin C (HbC) beta chain (SEQ ID NO. 7):

| | | | | | |
|---|---|---|---|---|---|
| 10 | 20 | 30 | 40 | 50 | 60 |
| MVHLTPKEKS | AVTALWGKVN | VDEVGGEALG | RLLVVYPWTQ | RFFESFGDLS | TPDAVMGNPK |
| 70 | 80 | 90 | 100 | 110 | 120 |
| VKAHGKKVLG | AFSDGLAHLD | NLKGTFATLS | ELHCDKLHVD | PENFRLLGNV | LVCVLAHHFG |
| 130 | 140 | | | | |
| KEFTPPVQAA | YQKVVAGVAN | ALAHKYH | | | |

Two α-chains and two β-chains constitute normal adult hemoglobin, which comprises about 97% of total hemoglobin in adults. SCD is caused by a single amino acid substitution (glutamic acid to valine, Glu→Val) at the 6^{th} position of β-globin chain (146 amino acids) from the N-terminus (Table 2). The abnormal hemoglobin is referred to as sickle cell hemoglobin (HbS, FIGS. 3A-3D). Because there is only one amino acid difference between HbS, HbC, and HbA, screening for antibodies against this subtle mutation can be be difficult. Therefore, selecting an efficient approach to develop antibodies targeting this subtle difference in the epitope conformation of hemoglobin β-chain can be relevant to the success of an effective screening and diagnostic approach. In addition, the specificity and sensitivity of antibodies against HbS, HbC and HbA can be a factor since the quality and amount of antibodies determine the overall performance of a diagnostic or screening assay such as the disclosed LFIA. The instant disclosure provides for the development, production and use of such antibodies that can be used, for example, as capture antibodies against HbS, HbC, and HbA in a diagnostic and/or screening assay or device. To decrease variation that can in some instances be caused by different batches of polyclonal antibodies, the development of monoclonal capture antibodies is also provided herein. Monoclonal antibodies can in some aspects provide stable performance and maintain relatively high consistency in assay performance, for example. The monoclonal antibodies can in some aspects be screened and selected from the polyclonal antibodies designed and disclosed herein.

In some embodiments, provided herein are capture antibodies against human HbS, HbC and HbA, and a detection antibody against the C-terminus of human hemoglobin α-chain or β-chain. Such antibodies can in some embodiments be developed using short peptides as the corresponding immunogens. Particularly, in some embodiments the amino acid sequences of the immunogens for eliciting an immune response in a host and generating antibodies are illustrated in FIGS. 3A-3D. Three of them contain 14 amino acids spanning the mutation sites in HbA (FIG. 3A; SEQ ID NO. 1), HbS (FIG. 3B; SEQ ID NO. 2), and HbC (FIG. 3C; SEQ ID NO. 3). The fourteenth amino acid on each, as illustrated in FIGS. 3A-3C, is cysteine which in some embodiments is added to facilitate peptide conjugation. The fourth immunogenic peptide comprises 19 amino acids from the C-terminal region of β-globins to elicit production of a detection pAb (FIG. 3D; SEQ ID NO. 4). The first amino acid (or nineteenth from the C-terminal), as illustrated in FIG. 3D, is cysteine which in some embodiments is added to facilitate peptide conjugation.

Representative immunogens for HbA, HbS, and HbC antibodies (SEQ ID NOs. 1-3, respectively) include the wild-type or the mutant amino acid at the 6th position (depicted in gray in FIGS. 3A-3C) from the N-terminus of human hemoglobin β-chain. They also include the flanking regions of the sixth position (from the beginning to the thirteenth amino acid of the N-terminus of human hemoglobin β-chain). This antigen design not only elicits antibody recognizing the subtle changes in antigens such as the single amino-acid mutation in human hemoglobin β-chains, but also provides an appropriate length of amino acid sequence to induce host animal's immunity. The design of these antigens results in antibodies that are ideally suited for the disclosed SCD testing methods, systems and devices.

Thus, in some embodiments, provided herein are immunogenic peptides capable of eliciting human HbA, HbS and/or HbC specific polyclonal or monoclonal antibodies. The peptides can in some embodiments comprise the first 13 amino acids from the N-terminus of HbA, HbS or HbC, wherein the peptides can comprise an amino acid sequence selected from SEQ ID NO. 1, SEQ ID NO. 2, or SEQ ID NO. 3, respectively. In some embodiments, such immunogenic peptides can comprise a cysteine at the fourteenth amino acid to facilitate peptide conjugation.

Moreover, in some aspects provided herein is an immunogenic peptide capable of eliciting a human hemoglobin β-chain specific polyclonal antibody. In some embodiments, the peptide comprises the first 18 amino acids from the C-terminus of human hemoglobin β-chain, wherein the peptide can comprise an amino acid sequence of SEQ ID NO. 4. In some embodiments, such an immunogenic peptide can further comprise a cysteine at the first amino acid to facilitate peptide conjugation. In some embodiments, the resulting antibody has an affinity for the amino acid region near the C-terminus, which is spatially distal from the sixth amino acid position near the N-terminus where the capture antibody binds. Therefore, the binding sites of the capture antibodies and the detection antibody do not overlap.

To develop monoclonal detection antibodies, in some embodiments provided herein antibodies can be designed and selected to detect the C-terminal region of human hemoglobin α-chain (α-globin). All hemoglobin should have human hemoglobin α-chain (e.g. HbA/S/C-α2β2, HbF-α2γ2, HbA2-α2δ2). Therefore, the detection antibody could carry all types of hemoglobin and potentially detect HbF and HbA2.

In some embodiments, these synthetic peptides (90% pure), as depicted in FIGS. 3A-3D, can be conjugated to potent immunogenic proteins, such as for example KLH, OVA and Imject™ Blue Carrier™ Protein (Thermo Fisher Scientific, Inc., Waltham, Massachusetts, United States of America), to elicit antibody production in a host. In some embodiments, the peptide antigens can also be conjugated to BSA for screening and evaluation purposes. In some embodiments, the host can for antibody production can be any animal suitable for antibody production and harvest, such as for example rabbits. The host can receive multiple immunizations, such as for example 5 injections with each immunogenic peptide. Polyclonal antisera titer can then be quantified by standard ELISA against each of the corresponding BSA-conjugated immunogenic peptides.

In some embodiments a method for generating a polyclonal or monoclonal antibody having an affinity to human HbC, HbS or HbA is provided. Such a method can comprise administering an immunogenic peptide, such as but not limited to a peptide selected from SEQ ID NO. 1, SEQ ID NO. 2, or SEQ ID NO. 3 to a subject or host, and collecting antibodies generated in the subject or host. Likewise, a method of generating a polyclonal or monoclonal antibody having an affinity to human hemoglobin α-chain or β-chain is also provided. Such a method can comprise administering an immunogenic peptide, such as but not limited to a peptide of SEQ ID NO. 4 to a subject or host, and collecting antibodies generated in the subject or host. In some embodiments, the immunogenic peptides administered to a subject or host can comprise an amino acid sequence that is substantially identical to, or has a sequence identity of about 60%, about 70%, about 80%, about 85%, about 90%, about 91%, about 92%, about 93%, about 94%, about 95%, about 96%, about 97%, about 98%, or about 99%, to that of SEQ ID NOs. 1-4.

Highly sensitive and specific capture and detection antibodies have been generated using the above. β-globin concentration ranges from about 6-44 μg/μL which is about 10-20% of the total hemoglobin concentration (6-22 g/dL) in newborns. As such, the microgram per milliliter level sensitivity of the disclosed antibodies, including as employed in the disclosed LFIA devices and methods, are sufficient to visually detect β-globin.

In some embodiments the antibodies disclosed and used herein, particularly detector antibodies for example, can be conjugated to a detectable moiety. In some embodiments, the detectable moiety can comprise a nanoparticle (NP), and particularly a detectable nanoparticle. In some embodiments, the detectable moiety and/or nanoparticle can comprise an enzyme label, a fluorescent label, a radiolabel, a particulate label, a colloidal gold label, a colored latex particles, or a phosphor converting label.

### Methods and Kits for Screening for and/or Assisting in Diagnosing Hemoglobinopathies

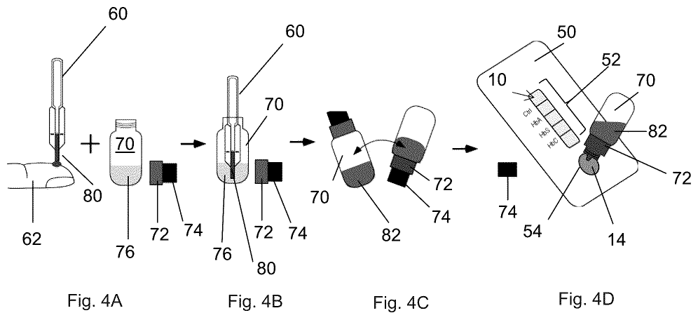

In some embodiments, methods are provided for screening for hemoglobinopathies. In some embodiments, methods are provided for diagnosing, and/or assisting in diagnosing, hemoglobinopathies. In some embodiments, such methods, as depicted in FIG. 4A for example, can comprise providing a blood sample (FIG. 4A), mixing the blood sample with a buffer (FIGS. 4B and 4C), providing a lateral flow immunoassay device 50 as disclosed herein, and loading the blood sample mixed in the buffer on the immunoassay device (FIG. 4D). If a hemoglobinopathy is present in the sample it will be revealed by the lateral flow immunoassay device.

Such methods are based, at least in part, on the ability to simultaneously detect the presence of HbS, HbC, and/or HbA in the sample. In some embodiments, these methods are based on, and/or provide for, the quantification of the levels of HbS, HbC, and/or HbA, in absolute or relative terms. Based on the simultaneous detection of HbS, HbC, and/or HbA in the sample, these methods can in some embodiments simultaneously differentiate sickle cell disease and trait, HbC disease and trait, and normal human hemoglobin.

The hemoglobinopathies that can be identified, screened for, and/or diagnosed, include HbAS, HbSS, HbSβ⁺-thalassemia, HbSβ⁰-thalassemia, sickle hemoglobin C disease (HbSC), and HbSX, wherein X is a globin chain variant that is not HbA, selected from the group consisting of HbSD-Punjab, HbSO-Arab, HbFS and HbSE. In some embodiments, the hemoglobinopathy can be a hemoglobin C disease or hemoglobin C trait, selected from the group consisting of HbC disease (HbCC), HbSβ⁰-thalassemia, HbSβ⁺-thalassemia, hemoglobin C trait (HbAC).

In some embodiments, the methods, as well as LFIA devices, disclosed herein can be based on a blood sample from a subject, e.g. a human subject. In some aspects, the blood sample can be selected from the group consisting of whole blood sample, dried blood sample, packed red cell sample, isolated or purified human hemoglobin protein sample, and freshly collected filter paper sample. A blood sample can be provided using any recognized and suitable approach, including for example finger-stick (FIG. 4A), heel-stick and/or venipuncture.

To prepare it for analysis using one of the disclosed methods, devices or systems, a blood sample can be mixed with a buffer comprising a detergent. In some embodiments, the buffer can be optimized to achieve hemolysis, dilution and conditioning of the sample. In some embodiments, the buffer, or extraction buffer, can comprise Brij 30, Tetronic 904, sodium borate and sodium azide, and have a pH of about 8.0. In some embodiments, the buffer can be optimized to lyse/hemolyze the red blood cells contained in the sample, release packed NP-detection antibodies conjugates, create an immunoreaction environment between detection antibodies and hemoglobins, and enhance particle movement on the strip. Once prepared, a sample that has been mixed in such a buffer can be loaded onto a LFIA device, for example, by adding the sample to a sample receiving area of the device.

In some embodiments, kits are provided for the detection of a hemoglobinopathy in a sample. FIGS. 4A to 4D illustrate an exemplary kit and steps for screening for a hemoglobinopathy in a sample. As illustrated in FIGS. 4A-4D, such a kit can comprise a LFIA device 50 or test strip as disclosed herein, a sampler 60 for collecting a blood sample, a buffer module 70 containing a buffer 76, and instructions for performing the detection of a hemoglobinopathy. In some aspects, the sampler 60 for collecting a blood sample 80, such as from a subject's finger 62 (FIG. 4A), can comprise a capillary tube as illustrated in FIGS. 4A to 4D. The buffer module 70 containing a buffer 76 can in some aspects comprise a two-piece cap having a lower portion 72 and upper portion 74. By removing both the lower portion 72 and upper portion 74 of the cap sampler 60 with the blood sample 80 can be inserted into buffer module 70 such that blood 80 can be added to buffer 76 (FIG. 4B).

Continuing with FIGS. 4A-4D, cap lower portion 72 can be reapplied to buffer module 70 and the contents, blood sample 80 and buffer 76, can be mixed (FIG. 4C) to form extract 82. After mixing, upper portion 74 of the cap can be removed such that an appropriate amount (e.g. 5 drops or about 100 μl) of the sample extract 82, or sample and buffer mixture, can be added to sample pad 14 by way of sample receiving area 54 of LFIA 50. Sample extract 82 will migrate or flow through test strip 10 and the results, i.e. presence or absence of HbS, HbC, and/or HbA, will be revealed in window 52.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a sample” includes a plurality of such samples, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods, compositions and apparatuses for detecting and/or diagnosing hemoglobinopathies, wherein one or more antibodies can be used directly, or in assays related thereto.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection.

As used herein, the terms “detectable moiety”, “detectable label”, and “detectable agent” refer to any molecule that can be detected by any moiety that can be added to a chemoprobe, antigen, inhibitor, marker, reagent and/or antibody, or a fragment or derivative thereof, that allows for the detection of the antigen, inhibitor, marker, reagent and/or antibody, fragment, or derivative in vitro and/or in vivo. Representative detectable moieties include, but are not limited to, dyes, chromophores, fluorescent moieties, radioacite labels, affinity probes, enzymes, antigens, groups with specific reactivity, chemiluminescent moieties, and electrochemically detectable moieties, etc. In some embodiments, the antibodies are biotinylated.

The subject(s) screened, tested, or from which a sample is taken, is desirably a human subject, although it is to be understood that the principles of the disclosed subject matter indicate that the compositions, apparatuses and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed compositions, apparatuses and methods are particularly useful in the testing, screening and/or treatment of warm-blooded vertebrates. Thus, the presently disclosed subject matter can in some embodiments concern mammals and birds.

More particularly, provided herein is the testing, screening and/or treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can have or be believed to have a hemoglobinopathy or related condition or phenotype.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Antibody Design and Production

The immunogenic peptides of FIGS. 3A-3D were synthesized (about 90% pure) and conjugated to potent immunogenic proteins (e.g. KLH, OVA, and Imject™ Blue Carrier™ Protein (Thermo Fisher Scientific, Inc., Waltham, Massachusettes, United States of America) to elicit antibody production. Some peptides were also conjugated to BSA for screening and evaluation purposes. Four rabbits were immunized by 5 injections with each immunogenic peptide. Polyclonal antisera titer was been quantified by standard ELISA against each of the corresponding BSA-conjugated immunogenic peptides.

To develop polyclonal detection antibody, the sequence covering the 142-145AA in the C-terminal region of human hemoglobin β-chain (β-globin) was designed and selected as the immunogenic peptide. The 142-145AA region is spatially distal from the 6th AA position near the N-terminus, the targeting site of the capture pAb. Therefore, the binding sites of the capture antibodies and the detection antibody do not overlap.

To develop monoclonal detection antibodies, the monoclonal antibody which detects the C-terminal region of human hemoglobin α-chain (α-globin) was selected. All hemoglobin should have human hemoglobin α-chain (e.g. HbA/S/C-α2β2, HbF-α2γ2, HbA2-α2δ2). Therefore, the detection antibody can carry all types of hemoglobin and potentially detect HbF and HbA2 for later design.

With the above innovative antibody designs, highly sensitive and specific capture and detection antibodies were generated. β-globin concentration ranges from about 6-44 μg/μL which is 10-20% of the total hemoglobin concentration (6-22 g/dL) in newborns. Based on experience, the sub-microgram level sensitivity of the disclosed LFIA should be sufficient to visually detect β-globin.

### Example 2

### Antibody Conjugation

Nanoparticles (NP) and other detectable labels/moieties were conjugated to the disclosed antibodies by activating the carboxy groups on the surface of nanoparticles and other detectable labels/moieties. The activated carboxy groups were then coupled with the amino groups of the hemoglobin pAbs or mAbs.

For the carboxy activation step, NP solution containing solid NP was mixed with MES buffer (50 mM MES, pH 6.0). To this suspension, EDAC (1-ethyl-3-(3-dimethylaminopropyl) carbodiimide hydrochloride, 20 mg/ml, stock solution was freshly prepared using MES buffer) was added. The mixture was rocked at room temperature for 30 minutes to form NP EDAC ester.

For the antibody coupling step, the mixture was centrifuged at 13,400 rpm for 10 minutes. The supernatant was then discarded. Then Borate buffer (20 mM, pH8.0) was mixed with the activated NP by vortexing and sonicating 10 minutes. The Ab was then added. The mixture was rocked for appropriate amount of time (1-4 hours) depending the detection antibody at room temperature. Blocking buffer (50 mM ethanolamine, 50 mg/mL BSA) was added to the solution and the mixture was rocked for another 30 minutes at room temperature. Then the mixture was centrifuged at 13,400 rpm for 10 minutes. The supernatant was discarded and an appropriate amount of HSTT buffer (including hepes, sucrose, trehalose, Tween-20, and Sodium Azide, pH 7.4) was added to resuspend the antibody-conjugated NPs.

### Example 3

### Design and Production of Multiplexed-Line Lateral Flow Immunochromatographic Assay Strip

Each polyclonal or monoclonal antibody against human HbC, HbS, HbA, and an antibody against the IgG of the host animal, from whom the detection antibody was generated were dispensed and immobilized on a chromatography matrix (such as, but not limited to, nitrocellulose). One end of this matrix was overlapped by a piece of absorbent pad (wick pad) and the other end of this matrix was overlapped by a conjugate pad. The multiplexed antibody lines on the matrix from a position near the absorbent pad to the other position near the conjugated pad were arranged as follows: Control line, HbA test line, HbS test line, and HbC test line. Of course, other configurations and/or orientations are equally applicable. The test antibodies in printing buffer (including sucrose, trehalose and sodium azide in PBS) were dispensed at the test lines on the chromatography matrix in amounts of about 20-25 ng/mm in lines with a width of about 0.5 mm. The chromatography matrix was dried at 50° C. for about 15 minutes. The colored or fluorescent nanoparticles conjugated with polyclonal or monoclonal detection antibody were sprayed at a dispense rate of about 1.25 μg/mm NPs in HSTT buffer, immunized on the conjugate pad and allowed to dry overnight at room temperature. A piece of sample pad was placed on top of the conjugate pad but not connected and touched to the chromatography matrix. The chromatography matrix, conjugate pad, sample pad, absorption pad, and the backing card were assembled and cut to a width of about 4 mm using a cutter so that the final strip had a dimension of about 4×60 mm. The manufactured strip was assembled in a disposable cassette and packed in a dehumidifier followed by storage at room temperature until use.

### Example 4

### Obtaining and Reagent Conditioning of a Blood Sample and Running MLFIA Test

The instant disclosure employs a sample for testing, with the principal sample source being human blood. Obtaining a blood sample can be easily performed via a finger-stick, heel-stick, venipuncture, blood bank samples, dry blood spot, or by using packed red blood cells. Blood obtained with a finger-stick or heel-stick can be collected in a capillary tube (FIGS. 4A-4D) or absorbed on filter paper, such as Whatman™ 903 Specimen Collection Paper designed for neonatal screening assays. A 3.2 mm (⅛″) punch obtained from the filter paper compares to about 2.0 to about 4.0 μl of whole blood depending on the amount of saturation of the whole blood into the paper. The reagent formula of an extraction buffer (including Brij 30, Tetronic 904, sodium borate and sodium azide, pH 8.0) was optimized to lyse/hemolyze the red blood cells contained in the collection step, release packed NP-detection antibodies conjugates, create immunoreaction environment between detection antibodies and hemoglobins, and enhance particle movement on the test strip. The amount of extraction buffer needed was optimized for the various Hb concentrations that will react with an optimized amount of the polyclonal or monoclonal detection antibody, which has been conjugated to colored or fluorescent nanoparticles. After collecting a certain amount (e.g. 5 μl) of whole blood sample, the test operator can remove the top two-piece vessel or module cap (FIG. 4A), immerse the capillary tube into the extraction buffer (FIG. 4B), and press on the capillary tube bulb and dispenses the blood into the buffer. If a whole blood sample is obtained from filter paper, the test operator only needs to immerse the filter paper into the buffer. Then the test operator can screw the two-piece cap together, invert the bottle gently (FIG. 4C), remove the top piece of the two-piece cap and drop or dispense an appropriate amount (e.g. about 100 μl) of the lysed whole blood sample into the MLFIA sample receiving area (FIG. 4D). The nanoparticle-conjugated detection antibody in the lateral flow test strip's conjugate pad can then bind to the C-terminal region of human hemoglobin α-chain or β-chain. Then the N-terminus of human hemoglobin β-chain will be captured by the polyclonal or monoclonal capture antibody in the corresponding S, C, or A test line. The nanoparticle-conjugated detection antibody will also be captured by the capture antibody immobilized at the control line to validate the test. The whole test can take about 1-5 minutes to complete.

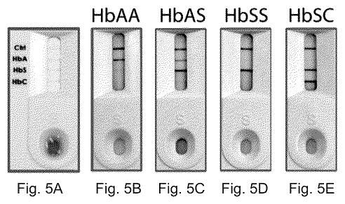

The presence of a color, in this case, blue line in the Ctrl window indicates that the test ran correctly. A blue line in the HbA, HbS, or HbC window indicates the presence of the respective hemoglobin (FIGS. 5A-5E). The disclosed LFIA device was used to screen samples from 137 human subjects. Of the 137 subjects the population comprised 30 normal (HbAA) subjects, 24 sickle trait (HbAS) subjects, 4 HbC disease trait (HbAC) subjects, and 79 SCD (42 HbSS and 37 HbSC) subjects. The LFIA test was able to detect individual genotypes listed above with sensitivity of >99% and specificity of >99% (Table 3). The ability to perform SCD screening, and ultimately diagnosis, at this level of accuracy is unprecedented among other point-of-care approaches, particularly for such a simple and inexpensive device.

TABLE 3
MLFIA test performance

| | SS | AS | SC | AC | AA | TOTAL |
|---|---|---|---|---|---|---|
| Clinical SS | 42 | 0 | 0 | 0 | 0 | 42 |
| Clinical AS | 0 | 24 | 0 | 0 | 0 | 24 |
| Clinical SC | 0 | 0 | 37 | 0 | 0 | 37 |
| Clinical AC | 0 | 0 | 0 | 4 | 0 | 4 |
| Clinical AA | 0 | 0 | 0 | 0 | 30 | 30 |
| TOTAL | 42 | 24 | 37 | 4 | 30 | 137 |
| Specificity | >99% | >99% | >99% | >99% | >99% | >99% |

### REFERENCES

1. Diallo D, Tchernia G. Sickle cell disease in Africa. Current opinion in hematology. 2002; 9 (2): 111-6. PubMed PMID: 11844993.
2. Wethers DL. Sickle cell disease in childhood: Part I. Laboratory diagnosis, pathophysiology and health maintenance. American family physician. 2000; 62 (5): 1013-20, 27-8. PubMed PMID: 10997528.
3. Weatherall D, Akinyanju O, Fucharoen S, Olivieri N, Musgrove P. Inherited Disorders of Hemoglobin. In: Jamison DT, Breman JG, Measham AR, Alleyne G, Claeson M, Evans DB, et al., editors. Disease Control Priorities in Developing Countries. 2nd ed. Washington (DC)2006.
4. Yang X, Kanter J, Piety NZ, Benton MS, Vignes SM, Shevkoplyas SS. A simple, rapid, low-cost diagnostic test for sickle cell disease. Lab on a chip. 2013; 13 (8): 1464-7. doi: 10.1039/c3Ic41302k. PubMed PMID: 23429713.
5. SHEVKOPLYAS SS, YANG X, WASHKO JK, PIETY NZ. Paper based diagnostic test. Google Patents; 2013.
6. Milligan C, Rees DC, Ellory JC, Osei A, Browning JA, Hannemann A, et al. A non-electrolyte haemolysis assay for diagnosis and prognosis of sickle cell disease. The Journal of physiology. 2013; 591 (Pt 6): 1463-74. doi: 10.1113/jphysiol.2012.246579. PubMed PMID: 23297308; PubMed Central PMCID: PMC3607166.
7. Kumar AA, Patton MR, Hennek JW, Lee SY, D'Alesio-Spina G, Yang X, et al. Density-based separation in multiphase systems provides a simple method to identify sickle cell disease. Proceedings of the National Academy of Sciences of the United States of America. 2014; 111 (41): 14864-9. doi: 10.1073/pnas.1414739111. PubMed PMID: 25197072; PubMed Central PMCID: PMC4205650.
8. Kumar AA, Mace CR, Whitesides GM, Wirth DF. Density-based separation of biological analytes using multiphase systems. Google Patents; 2012.

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for screening for hemoglobinopathies, comprising:
providing a blood sample;
mixing the blood sample with a buffer;
providing a lateral flow immunoassay device comprising a lateral flow test strip comprising a chromatography matrix, a capture antibody immobilized on the test strip, wherein the capture antibody has a binding affinity to human hemoglobin A (HbA), human sickle cell hemoglobin (HbS), or human hemoglobin C (HbC), and a conjugated detector antibody, wherein the detector antibody has a binding affinity to hemoglobin (Hb), wherein the detector antibody is conjugated to a detectable moiety; and
loading the blood sample mixed in the buffer on the immunoassay device,
wherein a hemoglobinopathy present in the sample will be revealed by the lateral flow immunoassay device.

2. The method of claim 1, wherein the presence of HbS, HbC, and/or HbA in the sample is simultaneously determined.

3. The method of claim 1, wherein the level of HbS, HbC, and/or HbA is quantified in absolute or relative terms.

4. The method of claim 1, comprising simultaneously differentiating sickle cell disease and trait, HbC disease and trait, and normal human hemoglobin, based on the presence of one or more of HbS, HbC, and/or HbA.

5. The method of claim 1, wherein the hemoglobinopathy is selected from the group consisting of: HbAS, HbSS, HbSβ⁺-thalassemia, HbSβ⁰-thalassemia, sickle hemoglobin C disease (HbSC), and HbSX, wherein X is a globin chain variant that is not HbA, selected from the group consisting of HbSD-Punjab, HbSO-Arab, HbFS and HbSE.

6. The method of claim 1, wherein the hemoglobinopathy is selected from a hemoglobin C disease or hemoglobin C trait, selected from the group consisting of HbC disease (HbCC), HbCβ⁰-thalassemia, HbCβ⁺-thalassemia, hemoglobin C trait (HbAC).

7. The method of claim 1, wherein the blood sample is selected from the group consisting of whole blood sample, dried blood sample, packed red cell sample, isolated or purified human hemoglobin protein sample, and freshly collected filter paper sample.

8. The method of claim 1, wherein providing a blood sample comprises finger-stick, heel-stick or venipuncture.

9. The method of claim 1, wherein mixing the blood sample with a buffer results in hemolysis, dilution and conditioning of the sample.

10. The method of claim 1, wherein loading the blood sample mixed in the buffer on the assay device comprises adding the sample to a sample receiving area of the device.

11. The method of claim 1, wherein the immunoassay device comprises a first capture antibody immobilized on the test strip, wherein the first capture antibody has a binding affinity to HbA; a second capture antibody immobilized on the test strip, wherein the second capture antibody has a binding affinity to HbS; and a third capture antibody immobilized on the test strip, wherein the third capture antibody has a binding affinity to HbC.

12. The method of claim 11, wherein the first, second and third capture antibodies are immobilized on the test strip in analyte capture zones.

13. The method of claim 12, wherein the immunoassay device further comprises a fourth capture antibody immobilized on the test strip in a fourth analyte capture zone, wherein the fourth capture antibody has an affinity to IgG of the host animal of the conjugate detector antibody.

14. The method of claim 13, wherein the fourth capture antibody serves as a control for the immunoassay.

15. The method of claim 12, wherein the analyte capture zones are rectangular shaped or circular shaped.

16. The method of claim 12, wherein the analyte capture zones are simplexed or multiplexed with one or more antibodies having an affinity for HbA, HbS, HbC, other hemoglobins (Hbs), or combinations thereof.

17. The method of claim 1, wherein the test strip further comprises a sample receiving area.

18. The method of claim 17, wherein the sample receiving area is configured to receive whole blood samples, dried blood samples, packed red cell samples, isolated or purified human hemoglobin protein samples, or freshly collected filter paper samples.

19. The method of claim 12, wherein the analyte capture zones are arranged on the test strip in a linear array parallel and substantially equidistant on chromatography matrix.

20. The method of claim 12, wherein the test strip further comprises a conjugate pad, wherein the conjugate pad comprises the conjugated detector antibody, optionally wherein the conjugated detector antibody is impregnated into the conjugate pad.

21. The method of claim 20, wherein the conjugate pad is located between the sample receiving area and the analyte capture zones.

22. The method of claim 1, wherein the conjugated detector antibody comprises a polyclonal or monoclonal detection antibody against human hemoglobin α-chain or β-chain.

23. The method of claim 1, wherein the detectable moiety of the conjugated detector antibody comprises an enzyme label, a fluorescent label, a radiolabel, a particulate label, a colloidal gold label, a colored latex particle, or a phosphor converting label.

24. The method of claim 1, wherein the chromatography matrix comprises a nitrocellulose membrane, polyvinylidene fluoride membrane, (charge-modified) nylon membrane, or polyethersulfone membrane.

25. The method of claim 1, wherein the lateral flow test strip comprises a component of a competition assay, an indirect assay or a sandwich assay.

26. The method of claim 1, wherein the immunoassay device is configured to be used at the point-of-care.
